(19) United States
(12) Patent Application Publication
(10) Pub. No.: US 2019/0275520 A1
(43) Pub. Date: Sep. 12, 2019

(54) FLOW-THROUGH MICROFLUIDIC METHODS AND DEVICES FEATURING MEMBRANE-PERTURBING SURFACE INTERACTIONS FOR INTRACELLULAR DELIVERY

(71) Applicant: MASSACHUSETTS INSTITUTE OF TECHNOLOGY, CAMBRIDGE, MA (US)

(72) Inventors: Martin P. STEWART, Cambridge, MA (US); Robert S. LANGER, Newton, MA (US); Klavs F. JENSEN, Lexington, MA (US); Xiaoyun DING, Cambridge, MA (US)

(73) Assignees: MASSACHUSETTS INSTITUTE OF TECHNOLOGY, CAMBRIDGE, MA (US); MASSACHUSETTS INSTITUTE OF TECHNOLOGY, CAMBRIDGE, MA (US)

(21) Appl. No.: 16/090,582

(22) PCT Filed: Mar. 31, 2017
(86) PCT No.: PCT/US2017/025560

§ 371 Date: Oct. 1, 2018

Related U.S. Application Data
(60) Provisional application No. 62/316,237, filed on Mar. 31, 2016.

(51) Int. Cl. B01L 3/00; C12N 15/87; C12M 3/06; C12M 1/42

(52) U.S. Cl. CPC B01L 3/502746; C12N 15/87; B01L 3/502761; C12M 23/16; C12M 35/04; C12M 35/08; B01L 2300/0851; B01L 2400/086; B01L 2400/088; B01L 2300/0816; B01L 2300/0887; B01L 2300/163; B01L 2300/161; B01L 2200/0663

(57) ABSTRACT

Methods and apparatus that facilitate membrane-perturbing surface interactions for delivering a payload to a variety of cell types without resulting in a substantial loss in cell viability or alteration of endogenous cellular functions. In one example, an intracellular delivery tool comprises a microfluidic device (10) which includes a microfluidic flow channel (12) containing fluid therein and a membrane perturbing surface (22), in fluid communication with the microfluidic flow channel (12), with a plurality of perturbing features disposed thereon. An exemplary intracellular delivery method includes flowing a fluid containing cells therein along a membrane perturbing surface having a plurality of perturbing features disposed thereon, and delivering nanomaterial across a membrane of the cells in the fluid during and after contact between the cells and the membrane perturbing surface.

### CROSS REFERENCE TO RELATED APPLICATIONS

This application claims priority to U.S. Provisional Application No. 62/316,237, filed Mar. 31, 2016, which is incorporated by reference herein in its entirety.

### STATEMENT OF GOVERNMENT SUPPORT

This invention was made with Government support under Grant No. RO1 GM101420 awarded by the National Institutes of Health. The Government has certain rights in the invention.

### BACKGROUND

Delivery of exogenous compounds and macromolecular cargo to the intracellular space is an important step in probing biological processes and engineering cellular function. Apart from fundamental research, effective intracellular delivery is important for cell engineering applications in medicine, including next generation therapeutics such as gene therapy and adoptive T cell transfer. Indeed, there is significant potential to address patient disease through ex vivo gene therapy of primary (i.e. patient-derived) hematopoietic stem cells (HSCs), immune cells (e.g., T-cells), and other somatic stem cell systems. Despite the importance of intracellular delivery in research and therapeutic applications, there are many challenges associated with the development and use of effective systems and techniques.

The challenge of intracellular delivery to mammalian cells can be viewed through the lens of two major parameters: cell type and target material. For example, some of the most exciting target cell types, such as stem cells and primary immune cells, are also the most difficult to effectively deliver. Furthermore, recent gene editing tools such as zinc finger nucleases (ZFNs), TALENs, and CRISPR/Cas9 systems require co-delivery of proteins and nucleic acids, which is ideally done without plasmid expression. These examples and others highlight the value of universal delivery systems that can introduce a wide variety of materials into diverse cell types.

Existing techniques for intracellular delivery to primary immune cells have limitations. For example, electroporation can result in considerable cellular toxicity and viral vectors are unable to infect non-proliferating cells, such as resting lymphocytes. Other delivery techniques, such as antibody conjugation to macromolecules or drugs, require specific antibodies for each cell type and distinct designs to efficiently deliver different the attached payloads. Furthermore, these conjugates are expensive to produce and have potentially immunogenic properties. Aptamer-siRNA chimeric RNAs have been shown to result in targeted gene knockdown in vivo, with minimal toxicity or immune activation. However, chimeric RNAs have only been used to deliver small RNAs and require identifying specific targeting aptamers for each cell of interest, therefore posing cost and production challenges similar to those associated with antibody conjugates. Advances in nanoparticle and liposome based technologies have resulted in improved intracellular delivery of drugs and antigens to phagocytic antigen presenting cells, such as dendritic cells and monocyte/macrophages. However, most of these methods lead to endosomal uptake of the payload, and only a very small proportion of the payload (estimated as about 1 to 2%) escapes from the endosome to the cytosol, a required step for biological activity of most payloads. Many of the above described techniques also result in accumulation of non-biodegradable packaging or delivery material in the cell, which can affect cell function.

### SUMMARY

In view of the challenges described above, the Inventors have recognized and appreciated a need in the art for effective intracellular delivery methods capable of delivering a broad range of payload material to a variety of cell types without resulting in a substantial loss in cell viability or alteration of endogenous cellular functions. Membrane disruption-based modalities are attractive candidates for universal delivery systems in vitro and ex vivo. Emerging microfluidic systems demonstrate potential to overcome conventional delivery barriers by facilitating direct, controlled disruption of the cell membrane. Prominent examples include nanoneedles and cell squeezing. Cell squeezing involves the rapid deformation of cells as they pass through microfabricated silicon constrictions that are approximately one-half to one-third of the cell's diameter. Although the exact size of membrane disruptions has not been quantified, diffusive delivery of a variety of macromolecular materials including proteins, nucleic acids, quantum dots, carbon nanotubes, and other nanomaterials to a wide variety of cell types has been demonstrated (See e.g., U.S. Patent Application Nos. 2014/0287509 and 2016/0193605; PCT Publication Nos. WO 2016/077761; WO 2016/115179; and WO 2017/008063, each of which is incorporated by reference in their entireties). However, a number of issues related to the application of cell squeezing remain unresolved. For example, the delivery efficiency is, at least in part, limited by the constriction diameter of a particular device. For a heterogeneous and/or asynchronous population of cells, it is unlikely that all cells within the population will be the same size. Therefore, in a heterogeneous cell population traversing a given constriction geometry, cells that are too large in relation to the constriction diameter may be lysed, while cells that are too small may not experience sufficient deformation to properly disrupt the membrane and allow for cargo delivery. The membrane perturbation mechanisms are thought to involve sequential steps of membrane tear formation and healing over timescales of microseconds to minutes and the subsequent diffusion of molecular cargo into the cytosol. However, there is a general lack of insight into the mechanisms that govern formation of membrane disruptions and the kinetics of resealing. Furthermore, the safety issues surrounding the feasibility of treating patient cells with microfluidic cell squeezing and other membrane disruption-based intracellular delivery techniques remain an open question. Thus the response of cells to microfluidic squeezing and other similar lab-on-chip based membrane disruption treatments, as well as the feasibility of the clinical application of these techniques, remain poorly defined.

Accordingly, the present invention provides microfluidic systems and devices comprising membrane perturbing surfaces, for inducing temporary perturbations in cell membranes such that a payload can pass through to the cytosol of the cell. The present invention further provides methods of using the devices and systems described herein to intracellularly deliver a payload to a cell or population of cells.

In some embodiments, the present invention provides a microfluidic device for causing temporary perturbations in a membrane of a cell suspended in a solution comprising at least one microfluidic flow channel, wherein the microfluidic flow channel comprises a channel wall comprising an inner surface; a cross-sectional channel geometry having a perimeter defined by the channel wall; and a perturbation zone comprising at least one membrane perturbing surface constituting a portion of the inner surface of the channel wall, the at least one membrane perturbing surface comprising at least one perturbation feature, wherein the one or more perturbation features facilitate the temporary perturbations of the cell membrane when the cell suspended in solution flows through the perturbation zone.

In some embodiments, the present invention provides a microfluidic device for causing temporary perturbations in a membrane of a cell suspended in a solution comprising at least one microfluidic channel, wherein the microfluidic channel comprises a channel wall having an inner surface; a cross-sectional geometry having a perimeter defined by the channel wall; and a perturbation zone comprising at least two membrane perturbing surfaces constituting a portion of the inner surface of the channel wall, the at least two membrane perturbing surface each comprising one or more perturbation features, wherein the one or more perturbation features facilitate the induction of temporary perturbations of the cell membrane when the cell suspended in solution flows through the perturbation zone.

In some embodiments, the present invention provides a microfluidic device for inducing temporary perturbations in a membrane of one or more cells suspended in a solution comprising a microfluidic flow channel, wherein the microfluidic channel comprises a channel wall comprising an inner surface; a cross-sectional geometry having a perimeter defined by the channel wall, wherein the cross-sectional geometry of the microfluidic flow channel is selected from the group consisting of a circle, an ellipse, an elongated slit, a rectangle, a square, a hexagon, and a triangle; and a perturbation zone comprising at least one membrane perturbing surface constituting a portion of the inner surface of the channel wall, wherein the at least one membrane perturbing surface comprises one or more perturbation features, wherein the one or more perturbation features comprise at least one of: (i) one or more physical perturbation features selected from the group consisting of nanospikes, microspikes, nanoteeth, microteeth, nanowires, nanotubes, rough abrasions, ridges, and microblocks, wherein the one or more physical perturbation features facilitates the inducing of a first perturbation in the membrane of the one or more cells by one of shearing, friction, and/or compression; and (ii) one or more chemical perturbation features selected from the group consisting of a detergent, a chemical compound, and a dangling chemical moiety, wherein the one or more chemical perturbation features facilitate the induction of a second perturbation in the membrane of the one or more cells.

In some embodiments, the present invention provides a method of intracellularly delivering a payload to a cell, the method comprising (a) flowing a cell suspension through a microfluidic channel, wherein the microfluidic channel comprises (i) a channel wall comprising an inner surface; (ii) a cross-sectional geometry having a perimeter defined by the channel wall; and (iii) a perturbation zone comprising at least two membrane perturbing surfaces constituting a portion of the inner surface of the channel wall, the at least two membrane perturbing surface each comprising one or more perturbation features, wherein the one or more perturbation features facilitate the induction of temporary perturbations of the cell membrane when the cell suspended in solution flows through the perturbation zone; and (b) incubating the cell suspension with the payload for a predetermined amount of time after the cell suspension passes through the microfluidic flow channel.

In some embodiments, the present invention provides a method of intracellularly delivering a payload to one or more cells in a cell suspension, the method comprising (a) flowing a cell suspension through a microfluidic flow channel, so as to induce temporary perturbations of a cell membrane of the one or more cells and thereby facilitate delivering the payload to the one or more cells, wherein the microfluidic flow channel comprises: (i) a channel wall comprising an inner surface; (ii) a cross-sectional geometry having a perimeter defined by the channel wall, wherein the cross-sectional geometry is selected from the group consisting of a circle, an ellipse, an elongated slit, a rectangle, a square, a hexagon, and a triangle; and (iii) a perturbation zone comprising at least one membrane perturbing surface constituting a portion of the inner surface of the channel wall, wherein the at least one membrane perturbing surface comprises one or more perturbation features, wherein the one or more perturbation features comprise at least one of: (1) one or more physical perturbation features selected from the group consisting of nanospikes, microspikes, nanoteeth, microteeth, nanowires, nanotubes, rough abrasions, ridges, and microblocks, wherein the one or more physical perturbation features facilitates induction of the temporary perturbations in the membrane of the one or more cells by one of shearing, friction, and/or compression; and (2) one or more chemical perturbation features selected from the group consisting of a detergent, a chemical compound, and a dangling chemical moiety, wherein the one or more chemical perturbation features facilitate the induction of the temporary perturbations in the membrane of the one or more cells; and (b) incubating the cell suspension with the payload for a predetermined amount of time after the cell suspension passes through the microfluidic flow channel.

In some embodiments of the present invention, the membrane perturbing surface comprises one or more physical perturbation features. In some embodiments, the physical perturbation features are selected from the group consisting of nanospike, microspikes, nanoteeth, microteeth, nanowires, nanotubes, rough abrasions, ridges, and microblocks. In some embodiments, the membrane perturbing surface comprises one or more chemical perturbation features. In some embodiments, the physical perturbation features induce perturbations in the cell membrane by mechanical force. In some embodiments, the mechanical force is one of shearing, friction, and/or compression.

In some embodiments of the present invention, the membrane perturbing surface comprises one or more physical perturbation features. In some embodiments, chemical perturbation features are selected from the group consisting of a detergent, a chemical compound, and a dangling chemical moiety. In some embodiments, the chemical perturbation features induce perturbations in the cell membrane through hydrophobic interactions, by binding to proteins or carbohydrate residues, or by amplifying molecular scale adhesion during passage. In some embodiments, the membrane perturbing surface comprises physical perturbation features or chemical perturbation features. In some embodiments, the membrane perturbing surface comprises physical perturbation features and chemical perturbation features.

In some embodiments, the cross-sectional channel geometry of the microfluidic channel is selected from the group consisting of a circle, an ellipse, an elongated slit, a rectangle, a rectangle, a square, a hexagon, and a triangle. In some embodiments, the cross-sectional geometry of the microfluidic flow channel comprises at least one cross-sectional channel dimension that permits the one or more cells suspended in the solution to pass through the microfluidic flow channel. In some embodiments, the cross-sectional channel dimension of the microfluidic flow channel is selected to increase interactions between the cell and the perturbation features.

In some embodiments, the microfluidic flow channel comprises cross-sectional channel dimension that is at least the size of a starting diameter of the cell. In some embodiments, the starting diameter of the cell is increased, such that an increased diameter of the cell is larger than the cross-sectional channel dimension of the microfluidic flow channel. In some embodiments, the increased diameter of the cell is at least 150%, 200%, 250%, or at least 300% the cross-sectional channel dimension of the microfluidic flow channel. In some embodiments, the cell is suspended in a hypotonic solution.

In some embodiments, the cross-sectional channel dimension of the microfluidic flow channel is selected such that the solution flows through the microfluidic flow channel at a velocity of about 0.01 μL/sec to about 10⁵ μL/sec. In some embodiments, the cross-sectional channel dimension of the microfluidic flow channel is selected such that the solution flows through the microfluidic flow channel at a velocity of about 10 μL/sec. In some embodiments, the cross-sectional channel geometry, the cross-sectional channel dimension, the number of membrane perturbing surfaces, and/or the perturbation features are selected to induce perturbations in the cell membrane large enough for a payload to pass through. In some embodiments, the cross-sectional channel geometry, the cross-sectional channel dimension, the number of membrane perturbing surfaces, and/or the perturbation features are selected to reduce the likelihood that the cell will die as a result of processing.

In some embodiments, the microfluidic device is made from injection molded plastic.

In some embodiments, the microfluidic device further comprises a cell driver selected from a group consisting of a pressure pump, a gas cylinder, a compressor, a vacuum pump, a syringe, a peristaltic pump, a pipette, a piston, a capillary actor, a human heart, a human muscle, and gravity.

In some embodiments, the entirety of the membrane perturbing surface comprises one or more membrane perturbing features. In some embodiments, the perturbation zone comprises at least two, at least three, at least four, at least five, or at least six membrane perturbing surfaces. In some embodiments, at least a portion of each of the membrane perturbing surfaces comprises one or more perturbing features. In some embodiments, the entirety of each of the membrane perturbing surfaces comprises one or more perturbing features.

In some embodiments, the microfluidic devices described herein comprise a plurality of microfluidics flow channels arranged in series or in parallel.

In some embodiments, the payload is present in the cell suspension before, during, and/or after flowing the cell suspension through the microfluidic flow channel. In some embodiments, the payload is present in the cell suspension before, during, and/or after flowing the cell suspension through the microfluidic flow channel. In some embodiments, the predetermined amount of time is at least 0.0001 seconds. In some embodiments, the predetermined amount of time is between about 0.0001 seconds and 1 week. In some embodiments, the predetermined amount of time is between about 0.0001 seconds and 2 days. In some embodiments, the predetermined amount of time is between about 0.0001 seconds and 60 minutes. In some embodiments, the predetermined amount of time is between about 0.0001 seconds and 20 minutes.

In some embodiments, the payload comprises a polynucleotide, a modified polynucleotide, a protein, a nucleoprotein, a small molecule, a carbohydrate, a lipid, an expression vector, a nanoparticle, a fluorescent molecule, a biologic, synthetic, organic, or inorganic molecule or polymer thereof. In some embodiments, the polynucleotide is a deoxyribonucleic acid (DNA) or a ribonucleic acid (RNA). In some embodiments, the DNA or RNA polynucleotide comprises one or more modified nucleotides that increase the stability and/or half-life of the DNA or RNA polynucleotide in vivo and/or in vitro. In some embodiments, the DNA is methylated DNA. In some embodiments, the RNA polynucleotide is a short-interfering RNA (siRNA), a short-hair pin RNA (shRNA), a micro RNA (miR), an antagomir. In some embodiments, the modified nucleic acid is a peptide nucleic acid, a mopholino, or a locked nucleic acid. In some embodiments, the nucleoprotein is a naturally occurring chromosome, a portion thereof, a nucleosome, or a nucleic acid molecule in physical contact with or covalently bound to a protein. In some embodiments, the payload comprises is a nucleic acid molecule and a protein. In some embodiments, the payload comprises a nucleic acid molecule and a small molecule, sugar, or polymer of biological, synthetic, organic, or inorganic molecules. In some embodiments, a dimension of the payload is between about 5 nm to about 20 nm.

It should be appreciated that all combinations of the foregoing concepts and additional concepts discussed in greater detail below (provided such concepts are not mutually exclusive) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTIONS OF THE FIGURES

The skilled artisan will understand that the figures provided and described herein are for illustration purposes, and that the drawings are not intended to limit the scope of the present invention in anyway. It is to be understood that in some instances various aspects of the invention may be shown in an exaggerated or enlarged manner to facilitate understanding of the invention. In the drawings, like reference characters generally refer to like features, functionally similar elements, and/or structurally similar elements represented throughout the various figures. The drawings are not necessarily to scale, and emphasis is instead placed upon illustrating the principles of the systems, devices, and methods described herein.

FIG. 1 illustrates a side view of an exemplary microfluidic device suitable for delivering a variety of molecules across a cell membrane and into a cell (e.g., intracellular delivery) through interactions of a cell with a perturbation zone, according to one inventive implementation.

FIG. 2 illustrates a view along a flow path of an exemplary perturbation zone comprising smooth surfaces, and suitable for use in combination with chemical perturbing features and integration with a microfluidic device such as the one shown in FIG. 1. The illustrated device can be used to intracellularly deliver one or more compounds to a cell or population of cells (illustrated by broken lines).

FIG. 3 illustrates a view along a flow path of an exemplary perturbation zone comprising rough surfaces, and suitable for integration with a microfluidic device such as the one shown in FIG. 1. The illustrated device can be used to intracellularly deliver one or more compounds to a cell or population of cells (illustrated by broken lines).

FIG. 4 illustrates a view along a flow path of an exemplary perturbation zone comprising nanoteeth, and suitable for integration with a microfluidic device such as the one shown in FIG. 1. The illustrated device can be used to intracellularly deliver one or more compounds to a cell or population of cells (illustrated by broken lines).

FIG. 5 illustrates a view along a flow path of an exemplary perturbation zone comprising microteeth, and suitable for integration with a microfluidic device such as the one shown in FIG. 1. The illustrated device can be used to intracellularly deliver one or more compounds to a cell or population of cells (illustrated by broken lines).

FIG. 6 illustrates a view along a flow path of an exemplary perturbation zone comprising nanospikes, and suitable for integration with a microfluidic device such as the one shown in FIG. 1. The illustrated device can be used to intracellularly deliver one or more compounds to a cell or population of cells (illustrated by broken lines).

FIG. 7 illustrates a view along a flow path of an exemplary perturbation zone comprising microspikes, and suitable for integration with a microfluidic device such as the one shown in FIG. 1. The illustrated device can be used to intracellularly deliver one or more compounds to a cell or population of cells (illustrated by broken lines).

FIG. 8 is a table showing illustrative fabrication strategies of various microfluidic devices described herein.

FIG. 9A-FIG. 9B illustrate scanning electron micrograph (SEM) images of exemplary perturbation features.

### DETAILED DESCRIPTION

The microfluidic systems, devices, and methods described herein are suitable for use in the intracellular delivery of a wide range of payload materials to a variety of cell types. In some aspects, the devices described herein utilize membrane perturbing surfaces comprising physical and/or chemical perturbation features to induce temporary perturbations in cell membranes. Incubation of perturbed cells in a solution containing a payload allows for the passage of the payload into the cytosol of the cell. The inventive implementations disclosed herein therefore provide compositions, apparatus, systems and methods that allow for intracellular delivery of payloads, independent of cell type and independent of the nature of the payload.

### A. Definitions

Unless specifically defined otherwise, all technical and scientific terms used herein shall be taken to have the same meaning as commonly understood by one of ordinary skill in the art (e.g., in cell culture, molecular genetics, and biochemistry).

As used in this specification and the appended claims, the singular forms “a,” “an” and “the” include plural references unless the content clearly dictates otherwise.

Throughout this specification, unless the context requires otherwise, the word “comprise,” or variations such as “comprises” or “comprising” refers to the inclusion of a stated element or integer or group of elements or integers but not the exclusion of any other element or integer or group of elements or integers. Further, the statement of numerical ranges throughout this specification specifically includes all integers and decimal points comprised within the stated range. For example, “0.2-5 mg” is a disclosure of 0.2 mg, 0.21 mg., 0.22 mg, 0.23 mg, 0.24 mg, 0.25 mg, etc., 0.3 mg, 0.4 mg, 0.5 mg, etc., 1.0 mg, 2.0 mg, 3.0 mg, etc., up to 5.0 mg.

In the descriptions and in the claims, phrases such as “at least one of” or “one or more of” or “and/or” may occur followed by a conjunctive list of elements or features. These phrases are intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used in this application, the terms “about,” “approximately,” and “substantially” are used as equivalents in the context of a numerical value or range and refer to a range of values that fall within 10% or less in either direction (greater than or less than) of the stated reference value unless otherwise stated or otherwise evident from the context. In certain embodiments, the terms “about,” “approximately,” and “substantially” refer to a range of values that fall within 10%, 9%, 8%, 7%, 6%, 5%, 4%, 3%, 2%, 1%, or less in either direction of the stated reference value. Any numerals used in this application with or without about/approximately are meant to cover any normal fluctuations appreciated by one of ordinary skill in the relevant art.

An “increase” refers to an increase in a value (e.g., increased intracellular delivery of a compound) of at least 1% as compared to a reference or control level. For example, an increase may include a 1, 2, 3, 4, 5, 6, 7, 8, 9, 10, 15, 20, 25, 30, 35, 40, 45, 50, 60, 70, 80, 90, 100, 150, 200, 250, 500, 1000% or more increase. An increase may also be represented as a fold change, e.g., 1.1, 1.2, 1.5, 2, 3, 4, 5, 6, 7, 8, 9, 10, 15, 20, 30 or more fold (e.g., 500, 1000 fold) higher than a reference or control level (e.g., a cell or population of cells that has not been treated with a microfluidics device described herein).

A “decrease” refers to a decrease in a value (e.g., decreased cell death) of at least 1% as compared to a reference or control level. For example, a decrease may include a 1, 2, 3, 4, 5, 6, 7, 8, 9, 10, 15, 20, 25, 30, 35, 40, 45, 50, 60, 70, 80, 90, 100, 150, 200, 250, 500, 1000% or more decrease. A decrease may also be represented as a fold change, e.g., 1.1, 1.2, 1.5, 2, 3, 4, 5, 6, 7, 8, 9, 10, 15, 20, 30 or more fold (e.g., 500, 1000 fold) lower than a reference or control level (e.g., a cell or population of cells that has not been treated with a microfluidics device described herein).

As used herein, a “solution” refers to any physiologically-compatible solutions, particularly standard buffers or standard physiologically-compatible buffered solutions used to suspend cells. A buffer can be any buffer commonly used in the art including, but not limited to, maleic acid, phosphoric acid, citric acid, malic acid, formic acid, lactic acid, succinic acid, acetic acid, pivalic acid, phosphoric acid, L-histidine, MES, bis-tris, MOPSO, PIPES, imidazole, MOPS, BES, TES, HEPES, DIPSO, TAPSO, TEA, NaCl, KCl, Na₂HPO₄, KH₂PO₄, Na₂CO₃, or NaHCO₃. Non-limiting examples of buffered solutions include phosphate buffered saline (PBS), and media commonly used in cell culture such as RPMI, DMEM, or IMDM. Surfactants can also be added to a solution in order to reduce clogging of the microfluidic flow channel during operation. Exemplary surfactants can include, but are not limited to, poloxamer, animal-derived serum, and albumin protein, among others.

As used herein, the term “cell” refers to a cell derived from any living organism (e.g., a prokaryotic cell or a eukaryotic cell). In some embodiments, a cell is a mammalian cell, a bacterial cell, or an insect cell. In some embodiments, a cell is a mammalian cell such as a stem cell (e.g., embryonic stem cells, induced pluripotent stem cells (iPSCs) and the like), a red blood cell, a white blood cell (e.g., an immune cell such as a T cell, a B cell, a macrophage, a dendritic cell, a mast cell, a basophil, a neutrophil, an innate lymphocyte, a natural killer (NK) cell), or a cell derived from any mammalian tissue. Use of the term “cell” further encompasses populations of cells (e.g., more than one cell). Cells comprised within a population may be the same cell type (e.g., a homogenous cell population) or different cell types (e.g., a heterogeneous population) from one another, and/or may be a different states of differentiation and/or maturation. In some embodiments, a cell population may be comprised of cells of different sizes.

Certain exemplary embodiments are described herein to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments, and that the features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

### B. Microfluidic Devices

As used herein, the term “microfluidics system” refers to systems in which low volumes (e.g., μL, nL, pL, fL) of fluids are processed to achieve the discrete treatment of small volumes of liquids. Certain implementations described herein include multiplexing, automation, and high throughput screening. The fluids (e.g., a buffer, a solution, a payload-containing solution, or a cell suspension) can be moved, mixed, separated, or otherwise processed. In certain embodiments described herein, microfluidics systems are used to induce perturbations (e.g., holes) in the cell membrane that allow a payload or compound to enter the cytosol of the cell. In some embodiments, the microfluidics systems described herein comprise a microfluidics device.

As used herein, a microfluidic device refers to a device comprising one or more microfluidics channels wherein the device is capable inducing temporary disruptions in a cell membrane and resulting in the cellular uptake of a payload that is present in the surrounding solution. The terms “microfluidic channel” and “microfluidics flow channel” are used interchangeably herein and refer to a channel comprised within a microfluidics device through which a cell suspended in a solution (e.g., a cell suspension) can pass through. In some embodiments, the microfluidics channels described herein comprise an inlet, an outlet, a channel wall comprising an internal surface, and a perturbation zone comprising one or more perturbation features. For example, a cell suspension can enter the microfluidic device via an inlet of a microfluidics flow channel, pass through the microfluidics channel and the perturbation zone, and exit the microfluidics device via an outlet of the microfluidics flow channel. Passage of a cell through a perturbation zone induces temporary disruptions in the plasma membrane of the cell. These temporary disruptions are referred to herein as “perturbations.” Perturbations created by the methods described herein are breaches in a cell that allow material from outside the cell to move into the cell. Non-limiting examples of perturbations include a hole, a tear, a cavity, an aperture, a pore, a break, a gap, or a perforation. The perturbations (e.g., pores or holes) created by the methods described herein are not formed as a result of assembly of protein subunits to form a multimeric pore structure such as that created by complement or bacterial hemolysins. The microfluidics devices described herein may also be referred to as “intracellular delivery tools.”

The terms “payload”, “cargo”, and “delivery material” are used interchangeably herein and encompass any material to be intracellularly delivered to a cell. Payloads can included, but are not limited to, proteins, small molecules, nucleic acids (e.g. RNA and/or DNA), modified nucleic acids, lipids, carbohydrates, macromolecules, vitamins, natural and synthetic molecules and polymers thereof, fluorescent dyes and fluorophores, carbon nanotubes, quantum dots, nanoparticles, expression vectors, nucleoproteins, organic and inorganic molecules and polymers thereof, and steroids. In some embodiments, a payload comprises a nucleic acid and a protein. In some embodiments, a payloads comprises a gene editing system such as TALENs, CRISPR/Cas9, and zinc-finger nucleases.

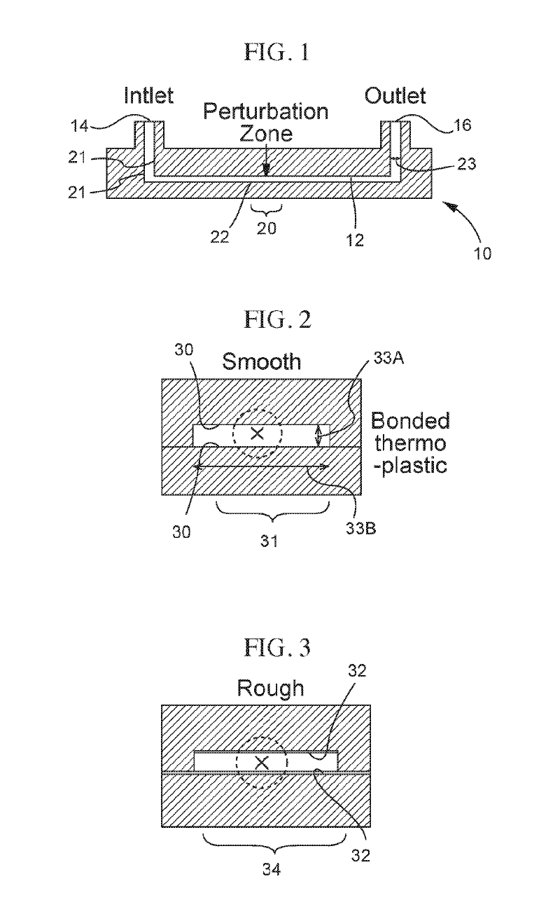

FIG. 1 shows an illustrative embodiment of a microfluidic device for delivering a payload across a membrane and into a cell for various treatment, engineering, and/or research purposes. In some embodiments, the microfluidic device 10 comprises a microfluidic flow channel 12. The flow channel 12 comprises an inlet 14, an outlet 16, and a channel wall 24. The flow channels comprised within the devices described herein further comprise a cross-sectional channel dimension 23 and a perturbation zone 20 comprising a membrane perturbing surface 22. The membrane perturbing surface 22 comprise one or more perturbation features (See e.g., FIGS. 2-6).

The microfluidic flow channels and perturbation zones comprised therein of the devices described herein comprise a channel wall (See e.g., FIG. 1, 24) comprising an internal surface, a cross-sectional channel geometry, and a cross-sectional dimension. The cross-sectional channel geometry of the microfluidic flow channel and/or perturbation zone comprises a perimeter that is defined by the channel wall and that may be of a variety of different shapes. In some embodiments, a cross-sectional channel geometry of the microfluidic flow channel is a circle, an ellipse, an elongated slit, a rectangle, a square, a hexagon, or a triangle. The cross-sectional dimensions of the microfluidic flow channel and/or perturbation zones can refer to a diameter, a length, a width, a depth, and/or a height (See e.g., FIG. 2 33A, 33B). For example, a microfluidic flow channel and/or perturbation zone comprising a circular cross-sectional geometry comprises a cross-sectional dimension of a diameter. Further, a microfluidic flow channel and/or perturbation zone comprising a square or rectangular cross-sectional geometry comprises the cross-sectional dimensions of a height, a width, and a depth. In some embodiments, the cross-sectional dimensions of a microfluidic flow channel and/or perturbation zone are substantially equal to or greater than a diameter of a cell in which perturbations are induced. Such cross-sectional dimensions do not substantially constrict the cells as the cells pass through the microfluidic flow channel and/or perturbation zone. In some embodiments, the microfluidic flow channel and/or perturbation zone comprises a cross-sectional dimension that is at least 101% a diameter of a cell in which perturbations are induced (e.g., the cross-sectional dimension is 1% larger than the diameter of the cell). In some embodiments, the microfluidic flow channel and/or perturbation zone comprises a cross-sectional dimension that is greater than 101% of a diameter of a cell in which perturbations are induced. For example, the cross-sectional dimension of the microfluidic flow channel and/or perturbation zone may be 101%, 105%, 110%, 115%, 120%, 125%, 130%, 140%, 150%, 160%, 170%, 180%, 190%, 200%, 225%, 250%, 300% or greater a size of a diameter of a cell in which perturbations are induced. In some embodiments, the microfluidic flow channel and/or perturbation zone comprises a cross-sectional dimension that is between about 1 μm to about 50 μm. For example, the microfluidic flow channel and/or perturbation zone may comprise a cross-sectional dimension that is about 1, 2, 3, 4, 5, 6, 7, 8, 9, 10, 11, 12, 13, 14, 15, 20, 25, 30, 35, 40, 45, or about 50 μm.

In some embodiments, the microfluidic flow channel and/or perturbation zone comprises a height that is between about 1 μm to about 50 μm. For example, the microfluidic flow channel may comprise a height that is about 1, 2, 3, 4, 5, 6, 7, 8, 9, 10, 11, 12, 13, 14, 15, 20, 25, 30, 35, 40, 45, or about 50 μm. In some embodiments, the microfluidic flow channel and/or perturbation zone comprises a width that is between about 1 μm to about 50 μm. For example, the microfluidic flow channel may comprise a width that is about 1, 2, 3, 4, 5, 6, 7, 8, 9, 10, 11, 12, 13, 14, 15, 20, 25, 30, 35, 40, 45, or about 50 μm. In some embodiments, the microfluidic flow channel and/or perturbation zone comprises a depth that is between about 1 μm to about 50 μm. For example, the microfluidic flow channel may comprise a depth that is about 1, 2, 3, 4, 5, 6, 7, 8, 9, 10, 11, 12, 13, 14, 15, 20, 25, 30, 35, 40, 45, or about 50 μm. In some embodiments, the microfluidic flow channel and/or perturbation zone comprises a diameter that is between about 1 μm to about 50 μm. For example, the microfluidic flow channel may comprise a diameter that is about 1, 2, 3, 4, 5, 6, 7, 8, 9, 10, 11, 12, 13, 14, 15, 20, 25, 30, 35, 40, 45, or about 50 μm.

In some embodiments, the microfluidic flow channel comprises a length that is at least 1 μm. In some embodiments, the microfluidic flow channel comprises a length that is between about 1 μm to about 500 μm. For example, in some embodiments, the microfluidic flow channel comprises a length that is about 1, 5, 10, 15, 20, 25, 30, 35, 40, 45, 50, 100, 150, 200, 250, 300, 350, 400, 450, or about 500 μm. In some embodiments, the microfluidic flow channel comprises a length that is greater than 500 μm (e.g., 1000, 2000, 3000 μm or greater). In some embodiments, the perturbation zone comprises a length a length that is at least 1 μm. In some embodiments, the perturbation zone comprises a length that is between about 1 μm to about 500 μm. For example, in some embodiments, the perturbation zone comprises a length that is about 1, 5, 10, 15, 20, 25, 30, 35, 40, 45, 50, 100, 150, 200, 250, 300, 350, 400, 450, or about 500 μm. In some embodiments, the perturbation zone comprises a length that is greater than 500 μm (e.g., 1000, 2000, 3000 μm or greater). In some embodiments, the length of the microfluidic flow channel is equivalent to the length of the perturbation zone, such that the perturbation zone extends the through the entirety of the microfluidic flow channel. In some embodiments, the length of the microfluidic flow channel is greater than the length of the perturbation zone, such that the perturbation zone comprises a portion of the microfluidic flow channel.

In some embodiments, a cell is suspended in a solution, such as a hypotonic solution, that causes the cell to swell, thereby resulting in an increased diameter. In such embodiments, the cells comprise a starting diameter (e.g., the diameter of the cell in an unaltered state) and an increased diameter (e.g., the diameter of the cell as a result of cell swelling). In some embodiments, the increased diameter of the cell is at least 101% of the starting diameter of the cell (e.g., the increased diameter of the cell is 1% larger than the starting diameter of the cell). In some embodiments, the increased diameter of the cell is about 101% to 300% of the starting diameter of the cell. For example, the increased diameter of the cell may be about 101%, 105%, 110%, 115%, 120%, 125%, 130%, 140%, 150%, 160%, 170%, 180%, 190%, 200%, 225%, 250%, or about 300% of the starting diameter of the cell. This expansion can be caused through a variety of approaches, such as by placing the cell in a hypotonic buffer.

In some embodiments, a cross-sectional dimension of the microfluidic flow channel may be smaller than a diameter of the cell (e.g., small than an increased diameter or a starting diameter of a cell), such that the cells experience constriction as they flow through the microfluidic flow channel. In such embodiments, a cell comprises a starting diameter and a constricted diameter. In such embodiments, a constricted diameter of the cells may be about 20% to about 99% of the starting diameter of the cell. In these embodiments, constriction is not primarily to deform the cells but to force the cells into contact with perturbation features on the membrane perturbing surfaces.

In some embodiments, the present invention provides a microfluidic device that comprises a microfluidic flow channel comprising a perturbation zone. A “perturbation zone” refers to an area or segment of the microfluidic flow channel comprising at least one membrane perturbing surface comprising a plurality of one or more perturbing features. In some embodiments, the cross-sectional dimensions (e.g., the diameter, height, width, depth, and/or length) of the perturbation zone are equivalent to those of the microfluidic flow channel such that the entirety of the microfluidic flow channel may be considered a perturbation zone. In some embodiments, one or more of the cross-sectional dimensions of the perturbation zone are different than those of the microfluidic flow channel. In some embodiments, one or more cross-sectional dimensions of the perturbation zone may be greater than or less than the corresponding cross-sectional dimensions of the microfluidic flow channel. In some embodiments, the height of the perturbation zone may be greater than or less than the height of the microfluidic flow channel. In some embodiments, the length of the perturbation zone may be less than the length of the microfluidic flow channel. In some embodiments, the width of the perturbation zone may be greater than or less than the width of the microfluidic flow channel. In some embodiments, the depth of the perturbation zone may be greater than or less than the depth of the microfluidic flow channel. In some embodiments, one or more of the diameter, height, width, depth, and/or length of the perturbation zone are greater than the corresponding dimension of the microfluidic flow channel. In some embodiments, one or more of the diameter, height, width, depth, and/or length of the perturbation zone are less than the corresponding dimension of the microfluidic flow channel.

In some embodiments, the perturbation zone comprises at least one membrane perturbing surface. The membrane perturbing surfaces of the present invention constitute a portion of the inner surface of the channel wall and comprise one or more perturbation features that are capable of inducing temporary perturbations in a cell membrane. In some embodiments, the at least one membrane perturbing surface comprises one or more perturbation features. In some embodiments, the at least one membrane perturbing surface comprises a plurality of perturbation features. In some embodiments, the perturbation features are dispersed over a portion of a membrane perturbing surface. In some embodiments, the perturbation features are dispersed over the entirety of a membrane perturbing surface. In some embodiments, each surface of a perturbation zone is a membrane perturbing surface, wherein one or more perturbation features are dispersed over at least a portion of each membrane perturbing surface. For example, in some embodiments, a perturbation zone comprises 2, 3, 4, 5, or 6 or more membrane perturbation features. In some embodiments, the perturbation features are dispersed over the entirety of each membrane perturbing surface.

In some embodiments, the perturbing features are physical perturbing features. In some embodiments, physical perturbing features induce temporary perturbations in a cell membrane as a result of the physical (e.g., mechanical) force produced when the cell comes in contact with such a feature. In some embodiments, physical perturbing features induce temporary perturbations in a cell membrane as a result of shearing, friction, or compression forces. In particular embodiments, the physical perturbing features are comprised of the same material from which the microfluidic flow channel is made. In some embodiments, the physical perturbing features and the microfluidic flow channel comprise a single piece of material (e.g., the physical perturbing features are not separate pieces that otherwise attached to the microfluidic flow channel). In some embodiments, the physical perturbing features and the microfluidic flow channel comprise a two or more separate pieces of material, wherein the physical perturbing features are attached to the microfluidic channel by any means known in the art. In some embodiments, the perturbing features are selected from the group consisting of nanospikes, microspikes, nanoteeth, microteeth, nanotubes, nanowires, rough abrasions, ridges, and microblocks. Surface roughness of the membrane perturbing surfaces can be tuned by treating polymer surfaces with chemical treatments used for similar purposes currently known in the art.

In some embodiments, the perturbing features are chemical perturbing features. In some embodiments, chemical perturbing features are immobilized on a smooth membrane perturbing surface. In some embodiments, chemical perturbing features are immobilized on a membrane perturbing surface that also comprises one or more physical perturbing features. In some embodiments, the chemical perturbing features are selected from the group consisting of a detergent, a chemical compound, and a dangling chemical moiety. The chemical perturbing features can disrupt a membrane of a cell through a variety of ways, for example through hydrophobic interactions with components of cell membranes, by binding to proteins and/or carbohydrate residues on cell membranes, and/or by amplifying molecular scale adhesion/friction between the cell and the membrane perturbing surface during passage of the cell through the microfluidic channel. In some embodiments, the membrane perturbing surfaces comprise both physical and chemical perturbation features.

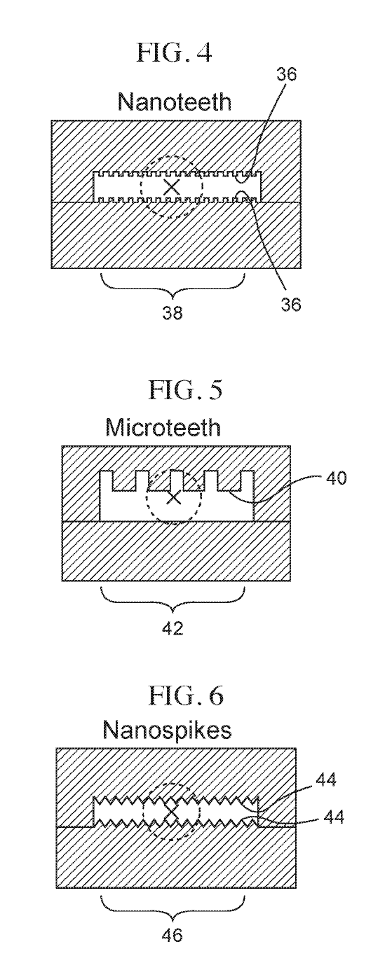

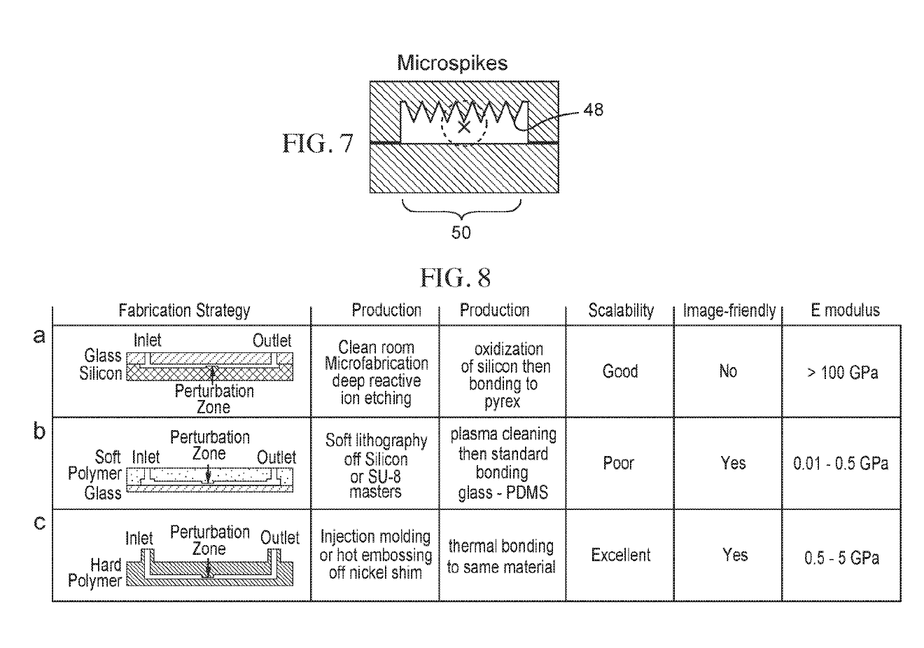

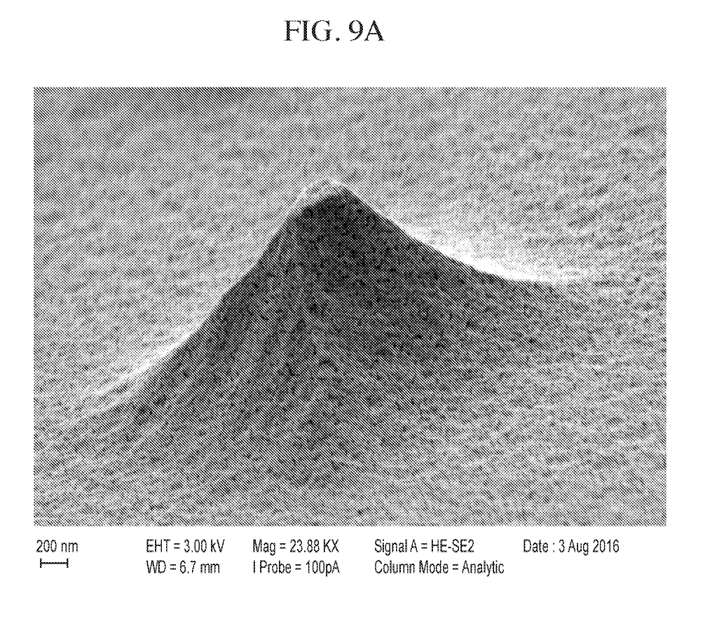

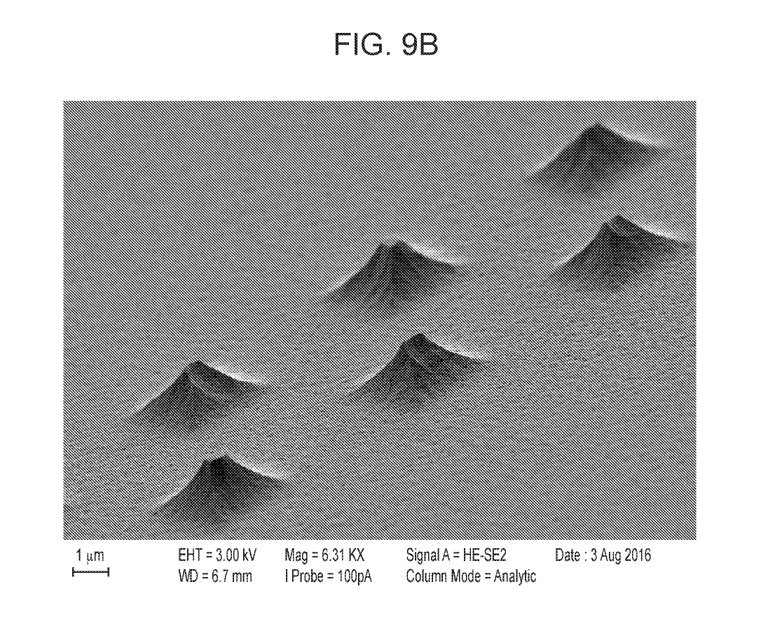

As shown in FIGS. 2-7, a variety of features can be used to disrupt or perturb the membrane of cells in the perturbation zone. For example, FIG. 2 shows a smooth surface 30 on two surfaces of a perturbation zone 31 on which chemical perturbing features can be disposed. FIG. 3 shows a rough surface 32 on two surfaces of a perturbation zone 34. FIG. 4 shows nanoteeth 36 disposed on two surfaces of a perturbation zone 38. FIG. 5 shows microteeth 40 formed on one surface of a perturbation zone 42. FIG. 6 shows nanospikes 44 formed on two surfaces of a perturbation zone 46. FIG. 7 shows microspikes 48 formed on one side of a perturbation zone 50. In addition to the preceding examples, perturbation features can be take any shape and be formed on one or more surfaces of a perturbing zone in a flow channel as long as the perturbation features are able to perturb membranes of cells passing along the perturbation features. Dimensions of the perturbation features can vary. For example, the physical perturbation features can have a height of about 0.001 μm to about 10 μm, a width of about 0.001 μm to about 10 μm, and/or a length of about 0.001 μm to about 500 μm.

In some embodiments, the perturbation zone comprises at least two membrane perturbing surfaces (e.g., two plates). In some embodiments, the perturbation zone comprises at least three, four, five, or six membrane perturbing surfaces. In some embodiments, each of the membrane perturbing surfaces comprises one or more perturbing features. In some embodiments, each of the membrane perturbing surfaces comprises a plurality of perturbing features.

In some embodiments, the microfluidic device and perturbing features comprised therein can be configured to cause temporary perturbations in cell membranes, thereby increasing the permeability of the cells. In such embodiments, the induced perturbation in the cell membrane are large enough for a payload to pass through to the intracellular space of the cell. In some embodiments, membrane perturbations of at least 0.1 nm are induced. In some embodiments, membrane perturbations of at least 1 nm are induced. In some embodiments, membrane perturbations between about 5 to about 20 nm are induced. In some embodiments, the perturbations induced in the cell membrane are sufficient in size and/or number to allow for the intracellular delivery of a payload, but do not result in a substantial loss of cell viability.

While a flow channel is shown in FIG. 1, a variety of approaches can be used to bring cells into contact with a membrane perturbing surface. For example, two or more surfaces can be arranged facing one another, such as plates positioned across from each other and allowing cells suspended in solution to flow there between. In other embodiments, cells can experience a flow trajectory that causes the cells to collide with a membrane perturbing surface. Thus cells can flow across a membrane perturbing surface in a variety of ways.

In some embodiments, the microfluidic devices described herein comprise a plurality of microfluidic channels. In some embodiments, the microfluidic devices described herein comprise at least 2 microfluidic channels. In some embodiments, the microfluidic devices described herein comprise at least 5, 10, 15, 20, 25, 50, 100, 150, 200, 300, 400, 500, 1000 or more microfluidic channels. In some embodiments, the plurality of microfluidic channels are arranged in series. In some embodiments, the plurality of microfluidic channels are arranged in parallel. In such embodiments, the architecture of the microfluidic devices can be structured to distribute cells evenly across all perturbation zones of the parallelized architecture to drive the cells through with a uniform flow rate and pressure. Baffles and channel splits can be added near the inlet or along the flow channel to ensure appropriate cell speed and distribution at the perturbation zone. Dimensions of the microfluidics channels can be modified or altered to allow low aspects ratio features that are more amenable to production of nickel masters commonly used for de-molding of injection molded plastics, and intricate nanoscale fabrication of the surfaces can be achieved that interface with and constrain (as needed) the passing cell.

In some embodiments, the microfluidics systems and/or devices further comprise a cell driver to facilitate the movement of cells through the device. Cells are moved (e.g., pushed or flowed) through the microfluidics channels by application of pressure. In some embodiments, said pressure is applied by a cell driver. As used herein, a cell driver is a device or component that applies a pressure or force to the buffer or solution in order to drive a cell through a constriction. In some embodiments, a pressure can be applied by a cell driver at the inlet. In some embodiments, a vacuum pressure can be applied by a cell driver at the outlet. In certain embodiments, the cell driver is adapted to supply a pressure greater than 90 psi. For example, the pressure supplied by the cell driver can be greater than 91, 92, 93, 94, 95, 100, 110, 120, 130, or 150 psi. In further embodiments, the cell driver is adapted to apply a pressure of 120 psi. In certain embodiments, the cell driver is selected from a group consisting of a pressure pump, a gas cylinder, a compressor, a vacuum pump, a syringe pump, a peristaltic pump, a pipette, a piston, a capillary actor, a human heart, human muscle, gravity, a microfluidics pumps, and a syringe. Modifications to the pressure applied by the cell driver also affect the velocity at which the cells pass through the microfluidics channel (e.g., increases in the amount of pressure will result in increased cell velocities).

Through these various approaches and structural designs of the microfluidic devices, the velocity (e.g., flow rate) of a solution (e.g., a cell suspension) through a microfluidic channel and along a membrane perturbing surface can be varied in a number of different ways based on various factors, such as the fluid being used, the cells contained within the fluid, and the molecules being delivered. In some embodiments, the solution moves through the microfluidic channel at a velocity of at least 0.01 μL/sec. In some embodiments, the solution moves through the microfluidic channel at a velocity of about 0.01 μL/sec to about 10⁵ μL/sec. In some embodiments, the solution moves through the microfluidic channel at a velocity of about 10 μL/sec.

As detailed in FIG. 8, the microfluidic device can be constructed through a variety of approaches. Thermoplastics can be used produced by injection molding. Materials employed can be polycarbonate and cyclic olefin copolymer (COO), both of which are FDA approved for food contact, medical devices and pharmaceutical packaging. These materials are also amenable to nanostructured fabrication via hot embossing and injection molding of robust shims, such as electroplated nickel masters used in the DVD industry for blu ray disc production. The construction approach can thus allow both the direct observation of cell behavior in the device with optical microscopy and the incorporation of nanostructured surface features (such as the nanospikes, nanopillars, controlled surface roughness, and various other textures) to affect membrane perturbation. Construction approaches can also be used based on silicon-glass bonded devices processed on the wafer scale. This production involves clean room processes for deep reactive ion etching of channel features followed by irreversible bonding of wafers to about 0.5 mm glass. The bonded devices can then be cut into individual units. These silicon-glass devices can be robust and highly reproducible. However, they are neither readily amenable to rapid prototyping nor compatible with imaging of the cell response by transmitted light optical microscopy. Other fabrication strategies can be used as well, such as using soft lithography of PDMS. This approach may lack sufficient young's modulus for effective perturbation of cells. Another strategy centers on injection molding or hot embossing of hard thermoplastics such as polycarbonate (PC), cyclic olefin copolymer (COC), and poly(methyl methacrylate) (PMMA). Nickel can be used by being electroplated onto a silicon master as a replica and then separated. The resulting nickel shim is durable and possesses favorable mechanical properties for the repeated pressing with melted thermoplastics. This approach can yield polymer devices with feature sizes down to about 50 nm and aspects ratios up to about 10:1. Such precision structures are almost impossible to produce with soft lithography, where the lateral resolution of PDMS is commonly cited at about 2 Furthermore, production by injection molding has excellent scalability with potential for low per unit production costs and relatively simple expansion to output levels appropriate for industrial processing. Apart from the production speed, which ranges from tens to hundreds of units per hour, a single nickel shim is durable enough to produce approximately 40,000 polymer units. The microfluidic device can also be made from injection molded plastic.

The perturbing features can be arranged and structured to result in an optimal level of membrane perturbation to allow intracellular delivery of various payloads. Perturbations that are too few in number or too small in size may result in a decrease in the efficiency of the intracellular delivery of the payload. Perturbations that are too many in number or too large in size may result in cell damage and/or death. In some embodiments, the microfluidic system, device can be configured to reduce the likelihood that the cell will die as a result of passing through the device and/or to optimize the level of intracellular delivery of a payload. Any of the components or aspects of the microfluidic system and/or device may be altered in order to optimize cell viability and/or level of intracellular delivery of a payload. For example, in some embodiments, the material the device is manufactured from, the nature of the perturbing features (e.g., physical or chemical perturbing features), the number of perturbing features on a membrane perturbing surface, the number of membrane perturbing surfaces, the dimensions of the microfluidic flow channel, the shape of the microfluidic flow channel, the nature of the solution the cells are suspended in, and/or the solution velocity may be modified to reduce the likelihood that the cell will die as a results of passing through the device. Further, perturbation zones and features can be characterized with SEM imaging and AEM topography to establish design features that govern optimal device architecture.

### C. Methods of Intracellular Delivery

In some embodiments, the present invention provides methods intracellularly delivering a variety of payloads into the cytosol of a cell. In some embodiments, the methods comprise providing the cell in a cell suspension, flowing the cell suspension through a microfluidic channel comprising a perturbation zone comprising one or more membrane perturbing surfaces, wherein the membrane perturbing surfaces comprise one or more perturbing features, and incubating the cell suspension with the payload for a predetermined amount of time after the cell suspension passes through the a microfluidic channel.

The methods of the present invention can be applied to a variety of cell types including, but not limited to, stem cells (e.g., embryonic stem cells, induced pluripotent stem cells (iPSCs) and the like), red blood cells, white blood cells (e.g., an immune cell such as a T cell, a B cell, a macrophage, a dendritic cell, a mast cell, a basophil, a neutrophil, an innate lymphocyte, a natural killer (NK) cell), or a cell derived from any mammalian tissue. The methods of the present invention can be applied to primary cells and/or cell lines. In some embodiments, the methods of the present invention allow for single-cell processing. In some embodiments, the methods of the present invention allow for the processing of a population of cells.

Effective intracellular delivery can be achieved by modulating physical and chemical properties of a surface that cells come in contact with during flow, for example as during flow through microfluidic channels, as well as the buffer composition, density of the cell suspension, and/or nature of the cell type. Further, the rate of membrane deformation and localization of forces at the cell surface can be major factors behind effective plasma membrane injury. However, additional effects provided by surface ligand presentation and extremely hydrophobic or hydrophilic interface chemistries can also affect effective plasma membrane injury to allow intracellular delivery. Substrates amenable to chemical functionalization can be used to form a flat side of a microfluidic device. The buffer composition can be hypotonic to swell cells to help ensure contact between any perturbing features and the cells. This approach can be applied to ex vivo treatment of patient cells in therapies targeted at a variety of different disorders, such as hematological diseases and blood disorders.

When a cell passes along the perturbing features, the cell undergoes rapid membrane disruption, which produces transient membrane disruptions of holes in the cell membrane. Molecules from the surrounding medium can then diffuse into the cell cytosol through these holes. After passing through the perturbation zone, the cell exits from the perturbing features and the holes can begin to close. The molecules can diffuse into the cell either during the cells' interaction with the perturbing features (while perturbation of the cell membrane is actively occurring) or after interaction between the cells and the perturbing features has finished (after active perturbation has ended but before the holes in the membrane close).

The effectiveness of various perturbation zones and perturbing features for intracellular delivery cell treatment can be experimentally determined by means known in the art. For example, the ability of a given device configuration to result in membrane perturbations sufficient for intracellular delivery of payloads and/or cell viability can be determined by flow cytometry, Western Blot, ELISA, PLA, immunohistochemistry, PCR, immunofluorescence, mass spectrometry, sequencing, microscopy, as well as single cell characterization approaches deployed in various mechanistic studies.

1. Compounds and Payloads

The present devices and methods allow for a broad range of payloads, including, nanoparticles, protein, quantum dots, RNA, and DNA, to be intracellularly delivered to almost any cell type, in high throughput. As used herein “payload” refers to a material that is being delivered to the cell. “Payload”, “cargo”, “delivery material”, and “compound” are used interchangeably herein. In some embodiments, a payload may refer to proteins, small molecules, nucleic acids (e.g. RNA and/or DNA), polynucleotides, modified polynucleotides, nucleoproteins, lipids, carbohydrates, macromolecules, vitamins, polymers, fluorescent dyes and fluorophores, carbon nanotubes, quantum dots, expression vectors, nanoparticles, steroids, and biologic, synthetic, organic, or inorganic molecules or polymers thereof.

In some embodiments, payload compositions such as polynucleotides, polypeptides, or other agents are purified and/or isolated. Specifically, as used herein, an “isolated” or “purified” nucleic acid molecule, polynucleotide, polypeptide, or protein, is substantially free of other cellular material or culture medium when produced by recombinant techniques, or chemical precursors or other chemicals when chemically synthesized. In some embodiments, purified compounds are at least 60% by weight (dry weight) the compound of interest. In some embodiments, the preparation is at least 75%, at least 90%, or at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. Examples of a an isolated or purified nucleic acid molecule include: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones.

In some embodiments, a payload comprises DNA and/or RNA. In some embodiments, DNA or RNA polynucleotide comprises one or more modifications that increase the stability and/or half-life of the DNA or RNA polynucleotide in vivo and/or in vitro. In some embodiments, the modified nucleic acid and/or polynucleotide is a peptide nucleic acid, a mopholino, or a locked nucleic acid. In certain embodiments, DNA or RNA can incorporate modified nucleotides, such as those with chemical modifications to the 2′-OH group in the ribose sugar backbone, such as 2′-O-methyl (2′OMe), 2′-fluoro (2′F) substitutions, and those containing 2′OMe, or 2′F, or 2′-deoxy, or “locked nucleic acid” (LNA) modifications.

In some embodiments the polynucleotide is an antisense polynucleotide, such as a short-interfering RNA (siRNA), a short-hair pin RNA (shRNA), a micro RNA (miR), or an antagomir. “Antisense” refers to a nucleic acid sequence, regardless of length, that is complementary to a nucleic acid sequence. Antisense RNA refers to single stranded RNA molecules that can be introduced to an individual cell, tissue, and/or organoid and results in decreased expression of a target gene through mechanisms that do not rely on endogenous gene silencing pathways. Antisense DNA refers to single stranded DNA molecules that can be introduced to an individual cell, tissue, or organoid and result in decreased expression of a target gene through mechanisms that do not rely on endogenous gene silencing pathways. An antisense nucleic acid can contain a modified backbone, for example, phosphorothioate, phosphorodithioate, or others known in the art, or may contain non-natural internucleoside linkages. Antisense nucleic acid can comprise, e.g., locked nucleic acids (LNA).

“Micro RNA” or “miRs” as used herein refers to a naturally occurring, small non-coding RNA molecule of about 21-25 nucleotides in length. miRs are at least partially complementary to one or more messenger RNA (mRNA) molecules. miRs can downregulate (e.g., decrease) gene expression through translational repression, cleavage of the mRNA, and/or deadenylation.

“Short hair-pin RNA” or “shRNA” are single stranded RNA molecules of about 50-70 nucleotides in length that form stem-loop structures and result in degradation of complementary mRNA sequences. shRNAs are encoded by DNA vectors that are introduced into cells via transfection or transduction and result in the integration of the shRNA-encoding sequence into the genome. As such, shRNA can provide stable and consistent repression of gene translation and expression.

“Small interfering RNA” or “siRNA” refers to a double stranded RNA. Optimally, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences.

“Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

As used herein, an “expression vector” is a DNA or RNA vector that is capable of affecting expression of one or more polynucleotides. Preferably, the expression vector is also capable of replicating within the host cell. Expression vectors can be either prokaryotic or eukaryotic, and are typically plasmids. Expression vectors of the present invention include any vectors that function (i.e., direct gene expression) in host cells of the present invention, including in one of the prokaryotic or eukaryotic cells described herein, e.g., gram-positive, gram-negative, pathogenic, non-pathogenic, commensal, cocci, bacillus, or spiral-shaped bacterial cells; archaeal cells; or protozoan, algal, fungi, yeast, plant, animal, vertebrate, invertebrate, arthropod, mammalian, rodent, primate, or human cells. Expression vectors of the present invention contain regulatory sequences such as transcription control sequences, translation control sequences, origins of replication, and other regulatory sequences that are compatible with the host cell and that control the expression of a polynucleotide. In particular, expression vectors of the present invention include transcription control sequences. Transcription control sequences are sequences which control the initiation, elongation, and termination of transcription. Particularly important transcription control sequences are those which control transcription initiation such as promoter, enhancer, operator and repressor sequences. Suitable transcription control sequences include any transcription control sequence that can function in at least one of the cells of the present invention. A variety of such transcription control sequences are known to those skilled in the art.

Depending on context, references herein to “vectors” may be distinguished from “expression vectors” in that “vectors” are intended to deliver a polynucleotide into a cell. For example, a virus is a vector which delivers viral DNA (an expression vector) into a target cell.

Additional payloads to be delivered via intracellular delivery can include a composition or complex including a nucleic acid molecule and a protein; a nucleic acid molecule covalently bound to a protein; a nucleic acid molecule in physical contact with or covalently bound to a protein, small molecule, sugar, or polymer of biological, synthetic, organic, or inorganic molecules; methylated DNA; a nucleic acid molecule wrapped around a protein; a composition including DNA and a histone; a naturally occurring chromosome or a portion thereof; an expression vector; a protein; a small molecule; a sugar; polymers of biological, synthetic, organic, or inorganic molecules; a charged molecule or composition comprising a charged molecule; an uncharged molecule; metabolites; membrane impermeable drugs; cryoprotectants; exogenous organelles; molecular probes; and/or nanodevices.

2. Methods of Use

Delivery of a variety of payloads can be achieved through the membrane microfluidic devices and methods provided herein, allowing for general intracellular delivery. The devices disclosed herein can be designed to be disposed of after a single use, or they can be designed to be used multiple times. In either case, however, the device can be reconditioned and used again after at least one use. Reconditioning can include any combination of the steps of disassembly of the device, followed by cleaning or replacement of particular pieces and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

In some embodiments, the device or components of the device are obtained and cleaned or sterilized. Cleaning and/or sterilization of the devices and/or components may be accomplished by any means known in the art including acid wash, bleach, ethanol, radiation, beta or gamma radiation, ethylene oxide, steam, autoclave, and/or a liquid bath (e.g., cold soak). An exemplary embodiment of sterilizing a device including internal circuitry is described in more detail in U.S. Patent Publication No. 2009/0202387, which is incorporated herein by reference in its entirety. In some embodiments, the instrument is placed in a closed and sealed container, such as a plastic or TYVEK bag. The container and instrument are then placed in a field of radiation that can penetrate the container, such as gamma radiation, x-rays, or high energy electrons. In some embodiments, the sterilized device is stored in a sterile container until use. It is preferred that the device, if implanted, is hermetically sealed. This can be done by any number of ways known to those skilled in the art.

In some embodiments, cells are incubated in a payload-containing solution or buffer for a period of time after undergoing processing with the microfluidics devices described herein. For example, cells can be incubated in a payload-containing solution for at least 0.0001 seconds. In some embodiments, cells are incubated in a payload-containing solution or buffer for a period of time between about 0.0001 seconds to about 1 week. In some embodiments, cells are incubated in a payload-containing solution or buffer for a period of time between about 0.0001 seconds to about 2 days. In some embodiments, cells are incubated in a payload-containing solution or buffer for a period of time between about 0.0001 seconds to about 60 minutes. In some embodiments, cells are incubated in a payload-containing solution or buffer for a period of time between about 0.0001 seconds to about 20 minutes. In certain embodiments, a payload is added to the cell suspension prior to processing with the microfluidics systems described herein. In certain embodiments, cells are processed as described herein while suspended a payload-containing solution or buffer.

In some embodiments, the devices and methods described herein are applied to regenerative medicine to enable cell reprogramming and/or stem cell differentiation. The current subject matter can be applied to immunology such as for antigen presentation and enhancement/suppression of immune activity through delivery to dendritic cells, monocytes, T cells, B cells and other lymphocytes. Further, imaging and sensing can benefit from improved delivery to target cells of quantum dots, carbon nanotubes and antibodies. Additionally, the current subject matter has application in cancer vaccines and research, such as for circulating tumor cell (CTC) isolation and lymphoma treatment. The method also provides a robust platform to screen for active siRNA and small molecule compounds capable of treating a disease or manipulating cell behavior.

The devices, technologies, and methods described herein may be implemented ex vivo, for example in a laboratory, centralized manufacturing facility, or cell-processing facility. In further embodiments, the devices, technologies, and methods are used as a bedside system in which patient samples, e.g., blood samples, are processed using a microfluidic device described herein, or a syringe adapted to include a microfluidic channel of appropriate size to deliver a payload to patient cells. Such a system is analogous to a bedside dialysis system.

In some embodiments, the devices and methods described herein provide greater precision and scalability of delivery when compared with prior techniques. For example, delivery of a material to a cell can be automated. Material such as proteins, RNA, siRNA, peptides, DNA, and impermeable dye can be implanted into a cell, such as embryonic stem cells or induced pluripotent stem cells (iPSCs), primary cells or immortalized cell lines. Further, the devices and methods described herein allow for proteins (especially large proteins, e.g., greater than 30, 50, 100, 150, 200, 300, 400, 500 kDa or more), quantum dots, or other payloads that are sensitive to or damaged by exposure to electricity, to be reliably delivered into cells while preserving the integrity and activity of the sensitive payload. Thus, the device and methods have significant advantages over existing techniques such as electroporation, which can damage the payload and leads to low cell viability. Another advantage of the present invention is that stem or precursor cells are rendered receptive to uptake of payload without altering the state of differentiation or activity of the treated cell. In addition to delivery of compositions into the cytoplasm of the cell for therapeutic purposes, e.g., vaccine production, the method is used to introduce molecules, e.g., large molecules comprising a detectable marker, to label intracellular structures such as organelles or to label intracellular constituents for diagnostic or imaging purposes.

In some embodiments, DNA can be delivered into cells such as primary stem cells and/or immune cells. Delivery of very large plasmids (even entire chromosomes) can be accomplished. Quantitative delivery into cells of known amount of a gene construct to study the expression level of a gene of interest and its sensitivity to concentration can also readily be accomplished. Delivery of known amounts of DNA sequences together with known amount of enzymes that enhance DNA recombination in order to achieve easier/more efficient stable delivery, homologous recombination, and site-specific mutagenesis can be accomplished. The methods and devices described herein can also be useful for quantitative delivery of RNA for more efficient/conclusive RNA studies. Delivery of small interfering RNA (siRNA) into the cytoplasm of a cell is also readily accomplished.

In some embodiments, quantitative delivery of drugs to cell models for improved screening and dosage studies can be achieved. The methods and devices described herein could be deployed as a high throughput method of screening protein activity in the cytosol to help identify protein therapeutics or understand disease mechanisms. Such applications are presently severely limited by current protein delivery methods due to their inefficiencies. The devices and techniques are useful for intracellular delivery of drugs to a specific subset of circulating blood cells (e.g. lymphocytes), high throughput delivery of sugars into cells to improve cryopreservation of cells, especially oocytes, targeted cell differentiation by introducing proteins, mRNA, DNA and/or growth factors, delivery of genetic or protein material to induce cell reprogramming to produce iPS cells, delivery of DNA and/or recombination enzymes into embryonic stem cells for the development of transgenic stem cell lines, delivery of DNA and/or recombination enzymes into zygotes for the development of transgenic organisms, DC cell activation, iPSC generation, and stem cell differentiation, nanoparticle delivery for diagnostics and/or mechanic studies as well as introduction of quantum dots. Skin cells used in connection with plastic surgery may also be modified using the devices and method described herein.

In some embodiments, the devices and methods described herein can be used to stimulate antigen presentation by delivering antigen and/or immune stimulatory molecules to antigen presenting cells, e.g., dendritic cells. In such embodiments, processed antigen presenting cells may demonstrate improved levels of activity compared to convention methods of stimulation, thereby leading to increased levels of T and B-cell mediated immunity to a target antigen. Such a method could thus be employed as a means of activating the immune system in response to cancer or infections.

In some embodiments, the devices and methods described herein can be used for screening, imaging, or diagnostic purposes by labeling cells. In such embodiments, a cell is labeled by intracellularly delivering a detectable marker such as a fluorescent molecule, a radionuclide, quantum dots, gold nanoparticles, or magnetic beads.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety. The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

The patent and scientific literature referred to herein establishes the knowledge that is available to those with skill in the art. All United States patents and patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

1. A microfluidic device comprising a microfluidic flow channel for inducing temporary perturbations in a membrane of one or more cells suspended in a solution, wherein the microfluidic flow channel comprises:
a. a channel wall comprising an inner surface;
b. a cross-sectional geometry having a perimeter defined by the channel wall, wherein the cross-sectional geometry of the microfluidic flow channel is selected from the group consisting of a circle, an ellipse, an elongated slit, a rectangle, a square, a hexagon, and a triangle; and
c. a perturbation zone comprising at least one membrane perturbing surface constituting a portion of the inner surface of the channel wall, wherein the at least one membrane perturbing surface comprises one or more perturbation features, and wherein the one or more perturbation features comprise at least one of:
i. one or more physical perturbation features selected from the group consisting of nanospikes, microspikes, nanoteeth, microteeth, nanowires, nanotubes, rough abrasions, ridges, and microblocks, wherein the one or more physical perturbation features facilitates inducing a first perturbation in the membrane of the one or more cells by one of shearing, friction, and/or compression; and
ii. one or more chemical perturbation features selected from the group consisting of a detergent, a chemical compound, and a dangling chemical moiety, wherein the one or more chemical perturbation features facilitates inducing a second perturbation in the membrane of the one or more cells.

2. The microfluidic device of claim 1, wherein the one or more perturbation features comprise the one or more physical perturbation features.

3. The microfluidic device of claim 1, wherein the one or more perturbation features comprise the one or more chemical perturbation features.

4. The microfluidic device of claim 3, wherein the one or more chemical perturbation features facilitate inducing the second perturbation in the cell membrane by at least one of:
at least one hydrophobic interaction;
binding to proteins or carbohydrate residues; and
amplifying molecular scale adhesion.

5. The microfluidic device of claim 1, wherein the one or more perturbation features comprise the one or more physical perturbation features and the one or more chemical perturbation features.

6. The microfluidic device of claim 5, wherein the one or more chemical perturbation features facilitate inducing the second perturbation in the cell membrane by at least one of:
at least one hydrophobic interaction;
binding to proteins or carbohydrate residues; and
amplifying molecular scale adhesion.

7. The microfluidic device of claim 6, wherein the cross-sectional geometry of the microfluidic flow channel comprises at least one cross-sectional channel dimension that permits the one or more cells suspended in the solution to pass through the microfluidic flow channel, such that the solution flows through the microfluidic flow channel at a velocity of between about 0.01 μL/sec to about 10⁵ μL/sec.

8. The microfluidic device of claim 7, wherein the one or more cells comprises a cell diameter and wherein the at least one cross-sectional channel dimension is equal to or greater than the cell diameter.

9. The microfluidic device of any one of claims 1-5, wherein the cross-sectional geometry of the microfluidic flow channel has at least one cross-sectional channel dimension that permits the one or more cells suspended in the solution to pass through the microfluidic flow channel such that the solution flows through the microfluidic flow channel at a velocity of between about 0.01 μL/sec to about 10⁵ μL/sec.

10. The microfluidic device of claim 9, wherein the one or more cells comprises a cell diameter and wherein the at least one cross-sectional channel dimension is equal to or greater than the cell diameter.

11. The microfluidic device of claim 7, wherein the cross-sectional channel dimension of the microfluidic flow channel is selected such that the solution flows through the microfluidic flow channel at a velocity of about 10 μL/sec.

12. The microfluidic device of claim 1, further comprising a cell driver, wherein the cell driver is selected from a group consisting of a pressure pump, a gas cylinder, a compressor, a vacuum pump, a syringe, a peristaltic pump, a pipette, a piston, a capillary actor, a human heart, a human muscle, and gravity.

13. The microfluidic device of any one of claims 1-12, wherein the microfluidic device comprises a plurality of microfluidics channels arranged in series or in parallel.

14. A method of intracellularly delivering a payload to one or more cells in a cell suspension, the method comprising:
a. flowing the cell suspension through a microfluidic flow channel, so as to induce temporary perturbations of a cell membrane of the one or more cells and thereby facilitate delivering the payload to the one or more cells, wherein the microfluidic flow channel comprises:
i. a channel wall comprising an inner surface;
ii. a cross-sectional geometry having a perimeter defined by the channel wall, wherein the cross-sectional geometry is selected from the group consisting of a circle, an ellipse, an elongated slit, a rectangle, a square, a hexagon, and a triangle; and
iii. a perturbation zone comprising at least one membrane perturbing surface constituting a portion of the inner surface of the channel wall, wherein the at least one membrane perturbing surface comprises one or more perturbation features, and wherein the one or more perturbation features comprise at least one of:
1. one or more physical perturbation features selected from the group consisting of nanospikes, microspikes, nanoteeth, microteeth, nanowires, nanotubes, rough abrasions, ridges, and microblocks, wherein the one or more physical perturbation features facilitates inducing the temporary perturbations in the membrane of the one or more cells by one of shearing, friction, and/or compression; and
2. one or more chemical perturbation features selected from the group consisting of a detergent, a chemical compound, and a dangling chemical moiety, wherein the one or more chemical perturbation features facilitate inducing the temporary perturbations in the membrane of the one or more cells; and
b. incubating the cell suspension with the payload for a predetermined amount of time after the cell suspension passes through the microfluidic flow channel.

15. The method of claim 14, wherein the one or more perturbation features comprise the one or more physical perturbation features.

16. The method of claim 14, wherein the one or more perturbation features comprise the one or more chemical perturbation features.

17. The method of claim 16, wherein the one or more chemical perturbation feature facilitates inducing the temporary perturbations in the cell membrane by at least one of:
at least one hydrophobic interaction;
binding to proteins or carbohydrate residues; and
amplifying molecular scale adhesion.

18. The method of claim 14, wherein the one or more perturbation features comprise the one or more physical perturbation features and the one or more chemical perturbation features.

19. The method of claim 18, wherein the one or more chemical perturbation features facilitate inducing the temporary perturbations in the cell membrane by at least one of:
at least one hydrophobic interaction;
binding to proteins or carbohydrate residues; and
amplifying molecular scale adhesion.

20. The method of claim 14, wherein the cross-sectional geometry of the microfluidic flow channel comprises at least one cross-sectional channel dimension that permits the one or more cells suspended in the solution to pass through the microfluidic flow channel, such that the solution flows through the microfluidic flow channel at a velocity of between about 0.01 μL/sec to about 10⁵ μL/sec.

21. The method of claim 20, wherein the one or more cells comprises a cell diameter and wherein the at least one cross-sectional channel dimension is equal to or greater than the cell diameter.

22. The method of any one of claims 14-18, wherein the cross-sectional geometry of the microfluidic flow channel has at least one cross-sectional channel dimension that permits the one or more cells suspended in the solution to pass through the microfluidic flow channel such that the solution flows through the microfluidic flow channel at a velocity of between about 0.01 μL/sec to about 10⁵ μL/sec.

23. The method of claim 22, wherein the one or more cells comprises a cell diameter and wherein the at least one cross-sectional channel dimension is equal to or greater than the cell diameter.

24. The method of claim 20, wherein the cross-sectional channel dimension of the microfluidic flow channel is selected such that the solution flows through the microfluidic flow channel at a velocity of about 10 μL/sec.

25. The method of claim 14, further comprising a cell driver, wherein the cell driver is selected from a group consisting of a pressure pump, a gas cylinder, a compressor, a vacuum pump, a syringe, a peristaltic pump, a pipette, a piston, a capillary actor, a human heart, a human muscle, and gravity.

26. A microfluidic device for causing temporary perturbations in a membrane of a cell suspended in a solution comprising at least one microfluidic flow channel, wherein the microfluidic flow channel comprises:
a. a channel wall comprising an inner surface;
b. a cross-sectional channel geometry having a perimeter defined by the channel wall; and
c. a perturbation zone comprising at least one membrane perturbing surface constituting a portion of the inner surface of the channel wall, the at least one membrane perturbing surface comprising at least one perturbation feature, wherein the one or more perturbation features facilitate the temporary perturbations of the cell membrane when the cell suspended in solution flows through the perturbation zone.

27. The microfluidic device of claim 26, wherein the perturbation features are physical perturbation features and are selected from the group consisting of nanospike, microspikes, nanoteeth, microteeth, nanowires, nanotubes, rough abrasions, ridges, and microblocks.

28. The microfluidic device of claim 26 or 27, wherein the physical perturbation features induce perturbations in the cell membrane by mechanical force.

29. The microfluidic device of claim 28, wherein the mechanical force is one of shearing, friction, and/or compression.

30. The microfluidic device of claim 26, wherein the perturbing features are chemical perturbation features and are selected from the group consisting of a detergent, a chemical compound, and a dangling chemical moiety.

31. The microfluidic device of claim 26 or 30, wherein the chemical perturbation features induce perturbations in the cell membrane through hydrophobic interactions, by binding to proteins or carbohydrate residues, or by amplifying molecular scale adhesion during passage.

32. The microfluidic device of any one of claims 26-31, wherein the membrane perturbing surface comprises physical perturbation features or chemical perturbation features.

33. The microfluidic device of any one of claims 26-31, wherein the membrane perturbing surface comprises physical perturbation features and chemical perturbation features.

34. The microfluidic device of claim 26, wherein the cross-sectional channel geometry is selected from the group consisting of a circle, an ellipse, an elongated slit, a rectangle, a square, a hexagon, and a triangle.

35. The microfluidic device of claim 34, wherein the cross-sectional geometry of the microfluidic flow channel comprises at least one cross-sectional channel dimension that permits the one or more cells suspended in the solution to pass through the microfluidic flow channel.

36. The microfluidic device of claim 35, wherein cross-sectional channel dimension of the microfluidic flow channel is selected to increase interactions between the cell and the perturbation features.

37. The microfluidic device of claim 36, wherein the microfluidic flow channel comprises cross-sectional channel dimension that is at least the size of a starting diameter of the cell.

38. The microfluidic device of claim 37, wherein the starting diameter of the cell is increased, such that an increased diameter of the cell is larger than the cross-sectional channel dimension of the microfluidic flow channel.

39. The microfluidic device of claim 38, wherein the increased diameter of the cell is at least 150%, 200%, 250%, or at least 300% the cross-sectional channel dimension of the microfluidic flow channel.

40. The microfluidic device of claim 38 or 39, wherein the cell is suspended in a hypotonic solution.

41. The microfluidic device of claim 26, wherein the cross-sectional channel dimension of the microfluidic flow channel is selected such that the solution flows through the microfluidic flow channel at a velocity of about 0.01 μL/sec to about 10⁵ μL/sec.

42. The microfluidic device of claim 41, wherein the cross-sectional channel dimension of the microfluidic flow channel is selected such that the solution flows through the microfluidic flow channel at a velocity of about 10 μL/sec.

43. The microfluidic device of claim 26, wherein the cross-sectional channel geometry, the cross-sectional channel dimension, the number of membrane perturbing surfaces, and/or the perturbation features are selected to induce perturbations in the cell membrane large enough for a payload to pass through.

44. The microfluidic device of claim 43, wherein the cross-sectional channel geometry, the cross-sectional channel dimension, the number of membrane perturbing surfaces, and/or the perturbation features are selected to reduce the likelihood that the cell will die as a result of processing.

45. The microfluidic device of claim 26, wherein the microfluidic device is made from injection molded plastic.

46. The microfluidic device of any of claims 26-45, further comprising a cell driver selected from a group consisting of a pressure pump, a gas cylinder, a compressor, a vacuum pump, a syringe, a peristaltic pump, a pipette, a piston, a capillary actor, a human heart, a human muscle, and gravity.

47. The microfluidic device of claim 26, wherein the entirety of the membrane perturbing surface comprises one or more membrane perturbing features.

48. The microfluidic device of claim 26, wherein the perturbation zone comprises at least two, at least three, at least four, at least five, or at least six membrane perturbing surfaces.

49. The microfluidic device of claim 48, wherein at least a portion of each of the membrane perturbing surfaces comprises one or more perturbing features.

50. The microfluidic device of claim 48, wherein the entirety of each of the membrane perturbing surfaces comprises one or more perturbing features.

51. The microfluidic device of any one of claims 26-50, wherein the microfluidic device comprises a plurality of microfluidics flow channels arranged in series or in parallel.

52. A microfluidic device for causing temporary perturbations in a membrane of a cell suspended in a solution comprising at least one microfluidic channel, wherein the microfluidic channel comprises:
a. a channel wall having an inner surface;
b. a cross-sectional geometry having a perimeter defined by the channel wall; and
c. a perturbation zone comprising at least two membrane perturbing surfaces constituting a portion of the inner surface of the channel wall, the at least two membrane perturbing surface each comprising one or more perturbation features, wherein the one or more perturbation features facilitate the induction of temporary perturbations of the cell membrane when the cell suspended in solution flows through the perturbation zone.

53. A method of intracellularly delivering a payload to a cell, the method comprising:
a. flowing a cell suspension through a microfluidic channel, wherein the microfluidic channel comprises:
i. a channel wall having an inner surface;
ii. a cross-sectional geometry having a perimeter defined by the channel wall; and
iii. a perturbation zone comprising at least one membrane perturbing surface constituting a portion of the inner surface of the channel wall, the at least one membrane perturbing surface comprising one or more perturbation features, wherein the one or more perturbation features facilitate the induction of temporary perturbations of the cell membrane when the cell suspended in solution flows through the perturbation zone; and
b. incubating the cell suspension with the payload for a predetermined amount of time after the cell suspension passes through the microfluidic flow channel.

54. The method of claim 53, wherein the perturbation features are physical perturbation features and are selected from the group consisting of nanospike, microspikes, nanoteeth, microteeth, nanowires, nanotubes, rough abrasions, ridges, and microblocks.

55. The method of claim 54, wherein the physical perturbation features induce perturbations in the cell membrane by mechanical force.

56. The method of claim 55, wherein the mechanical force is one of shearing, friction, and/or compression.

57. The method of claim 53, wherein the perturbation features are chemical perturbation features and are selected from the group consisting of a detergent, a chemical compound, and a dangling chemical moiety.

58. The method of claim 57, wherein the chemical perturbation features induce perturbations in the cell membrane through hydrophobic interactions, by binding to proteins or carbohydrate residues, or by amplifying molecular scale adhesion during passage.

59. The method of any one of claims 53-58, wherein the membrane perturbing surface comprises physical perturbation features or chemical perturbation features.

60. The method of any one of claims 53-58, wherein the membrane perturbing surface comprises physical perturbation features and chemical perturbation features

61. The method of claim 53, wherein the cross-sectional channel geometry of the microfluidic flow channel is selected from the group consisting of a circle, an ellipse, an elongated slit, a rectangle, a square, a hexagon, and a triangle.

62. The method of claim 53, wherein the microfluidic flow channel comprises a cross-sectional channel dimension selected to increase interactions between the cell and the perturbation features.

63. The method of claim 62, wherein the cross-sectional channel dimension of the microfluidic flow channel is at least the size of a starting diameter of the cell.

64. The method of claim 63, wherein the starting diameter of the cell is increased, such that an increased diameter of the cell is larger than the cross-sectional channel dimension of the microfluidic flow channel.

65. The method of any one of claim 64, wherein the increased diameter of the cell is at least 150%, 200%, 250%, or at least 300% the cross-sectional channel dimension of the microfluidic flow channel.

66. The method of claim 63 or 64, wherein the cell is suspended in a hypotonic solution.

67. The method of claim 53, wherein the cross-sectional channel dimension of the microfluidic flow channel is selected such that the cell suspension flows through the microfluidic flow channel at a velocity of about 0.01 μL/sec to about 10⁵ μL/sec.

68. The method of claim 67, wherein the cross-sectional channel dimension of the microfluidic flow channel is selected such that the cell suspension flows through the microfluidic flow channel at a velocity of about 10 μL/sec.

69. The method of claim 53, wherein the cross-sectional channel geometry, the cross-sectional channel dimension, the number of membrane perturbing surfaces, and/or the perturbation features are selected to induce perturbations in the cell membrane large enough for a payload to pass through.

70. The method of claim 69, wherein the cross-sectional channel geometry, the cross-sectional channel dimension, the number of membrane perturbing surfaces, and/or the perturbation features are selected to reduce the likelihood that the cell will die as a result of processing.

71. The method of claim 53, wherein the microfluidic device is made from injection molded plastic.

72. The method of any of claim 53, further comprising a cell driver selected from a group consisting of a pressure pump, a gas cylinder, a compressor, a vacuum pump, a syringe, a peristaltic pump, a pipette, a piston, a capillary actor, a human heart, a human muscle, and gravity.

73. The method of claim 53, wherein the entirety of the membrane perturbing surface comprises one or more membrane perturbing features.

74. The method of claim 53, wherein the perturbation zone comprises at least two, at least three, at least four, at least five, or at least six membrane perturbing surfaces.

75. The method of claim 74, wherein at least a portion of each of the membrane perturbing surfaces comprises one or more perturbing features.

76. The method of claim 75, wherein the entirety of each of the membrane perturbing surfaces comprises one or more perturbing features.

77. The method of any one of claims 53-76, wherein the microfluidic device comprises a plurality of microfluidics flow channels arranged in series or in parallel.

78. The method of any one of claim 14-25 or 53-76, wherein the payload is present in the cell suspension before, during, and/or after flowing the cell suspension through the microfluidic flow channel.

79. The method of any one of claim 14-25 or 53-78, wherein the predetermined amount of time is at least 0.0001 seconds.

80. The method of claim 79, wherein the predetermined amount of time is between about 0.0001 seconds and 1 week.

81. The method of claim 80, wherein the predetermined amount of time is between about 0.0001 seconds and 2 days.

82. The method of claim 81, wherein the predetermined amount of time is between about 0.0001 seconds and 60 minutes.

83. The method of claim 82, wherein the predetermined amount of time is between about 0.0001 seconds and 20 minutes.

84. The method of any one of claim 14-25 or 53-83, wherein the payload comprises a polynucleotide, a modified polynucleotide, a protein, a nucleoprotein, a small molecule, a carbohydrate, a lipid, an expression vector, a nanoparticle, a fluorescent molecule, a biologic, synthetic, organic, or inorganic molecule or polymer thereof.

85. The method of claim 84, wherein the polynucleotide is a deoxyribonucleic acid (DNA) or a ribonucleic acid (RNA).

86. The method of claim 85, wherein the DNA or RNA polynucleotide comprises one or more modified nucleotides that increase the stability and/or half-life of the DNA or RNA polynucleotide in vivo and/or in vitro.

87. The method of claim 85, wherein the DNA is methylated DNA.

88. The method of claim 85, wherein the RNA polynucleotide is a short-interfering RNA (siRNA), a short-hair pin RNA (shRNA), a micro RNA (miR), an antagomir.

89. The method of claim 86, wherein the modified nucleic acid is a peptide nucleic acid, a mopholino, or a locked nucleic acid.

90. The method of claim 84, wherein the nucleoprotein is a naturally occurring chromosome, a portion thereof, a nucleosome, or a nucleic acid molecule in physical contact with or covalently bound to a protein.

91. The method of claim 84, wherein the payload comprises is a nucleic acid molecule and a protein.

92. The method of claim 84, wherein the payload comprises a nucleic acid molecule and a small molecule, sugar, or polymer of biological, synthetic, organic, or inorganic molecules.

93. The method of any one of claims 84-92, wherein a dimension of the payload is between about 5 nm to about 20 nm.
